(19) United States
(12) Patent Application Publication
(10) Pub. No.: US 2024/0298909 A1
(43) Pub. Date: Sep. 12, 2024

(54) Pulse Notification Bracelet Device and Method of Sending Pulse Notification

(71) Applicant: Joseph Pecora, Brooksville, FL (US)

(72) Inventor: Joseph Pecora, Brooksville, FL (US)

(21) Appl. No.: 18/594,259

(22) Filed: Mar. 4, 2024

Related U.S. Application Data
(60) Provisional application No. 63/450,780, filed on Mar. 8, 2023.

(51) Int. Cl. A61B 5/024; A61B 5/00; A61B 5/0245

(52) U.S. Cl. CPC A61B 5/02438; A61B 5/02416; A61B 5/0245; A61B 5/681; A61B 5/7405; A61B 5/746; A61B 5/7475

(57) ABSTRACT

A wearable device designed to notify third parties in the event of the death of the wearer of the wearable device is disclosed. The device can be worn around the wrist and features a band with a pulse detecting sensor embedded on the inner surface thereof. The sensor continuously monitors the wearer's pulse. In the absence of a pulse for a predefined duration, an e-sim within the band, preconfigured with contact details of designated third parties, automatically sends out a notification to indicate that the wearer has passed away. The notification includes pre-set or customizable content and may include a timestamp of the last detected pulse.

### CROSS-REFERENCE TO RELATED APPLICATION

The present application claims priority to, and the benefit of, U.S. Provisional Application No. 63/450,780, which was filed on Mar. 8, 2023, and is incorporated herein by reference in its entirety.

### FIELD OF THE INVENTION

The present invention generally relates to the field of wearable devices. More specifically, the present invention relates to a novel wristband or bracelet capable of notifying one or more third parties that the wearer has passed away. The device includes a pulse detecting sensor and when the pulse of the wearer is not detected for a certain period of time, an automatic notification is sent to a pre-determined individual. The device prevents people from passing away and being left alone for days, weeks, or even months before being discovered. Accordingly, the present disclosure makes specific reference thereto. Nonetheless, it is to be appreciated that aspects of the present invention are also equally applicable to other like applications, devices, and methods of manufacture.

### BACKGROUND

By way of background, many people live alone due to a lack of family and friends. In fact, in many societies, there is a growing trend of individuals, especially older adults, living alone. It is due to various factors such as increased individualism, a desire for privacy, the passing of a spouse, or families being geographically dispersed. When a person lives alone, there is often no one present to quickly notice if they have passed away. In contrast, individuals who live with family or in community settings are likely to be noticed of their absence or unresponsiveness much sooner.

Accordingly, it may take days, weeks, or even longer in order to discover the death of a person living alone. The delay can occur because people living alone do not have daily check-ins from neighbors, friends, or family. The discovery of the death often happens when there is a lack of response to phone calls, accumulation of mail, or other signs. The lack of regular communication of people living alone results in others not immediately noticing their absence or demise. Also, it is difficult for neighbors and friends to do regular welfare checks. People desire a device and system for individuals living alone that prevents them from passing away and being left alone for days, weeks, or even months at a time.

Therefore, there exists a long-felt need in the art for a device that can be worn as a bracelet by a wearer to notify a third-party in the event of the wearer's death. Additionally, there is a long-felt need in the art for a non-intrusive wearable device that is simple to use and detects the pulse of the wearer. Moreover, there is a long-felt need in the art for a wearable device that continuously detects the pulse of a wearer and sends an alert to one or more third parties when no pulse is detected. Further, there is a long-felt need in the art for a bracelet device that offers peace of mind to family members and friends by promptly notifying them when a person wearing the bracelet passes away. Furthermore, there is a long-felt need in the art for an innovative wearable device that prevents people from being left alone for days, weeks, or even months after passing away. Finally, there is a long-felt need in the art for a bracelet device that can be worn at all times and detects the absence of a pulse to send a notification to one or more contacts about the passing away of the wearer.

The subject matter disclosed and claimed herein, in one embodiment thereof, comprises a wearer death notification bracelet. The bracelet is in the form of a band that forms a closed loop with an opening for receiving the wrist of the wearer. The band has an inner surface and an outer surface, the inner surface touches the skin of the wearer when the band is worn by the wearer. A pulse detecting sensor is embedded within the inner surface of the band and is configured to detect the pulse of the wearer when the sensor touches the skin of the wearer. An e-sim is embedded within the band and is preconfigured with contact details of one or more third-party contacts for sending notifications upon the detection of an absence of a pulse in the user, wherein the absence of the pulse is detected continuously for a threshold time. In one embodiment, the bracelet sends the notification using cellular capabilities of a connected smartphone. Basically, e-sim works by letting you connect to a second (or third, or fourth) mobile network without swapping physical SIM cards, one phone, multiple network options. Once an e-sim profile is downloaded, a connection can be made to a multi-network club. An e-sim (or embedded SIM) is a chip in a mobile device that can act like a physical SIM card. Rather than inserting a new SIM card to join a new mobile network, you can instead download an e-sim profile.

In this manner, the death notification wearable device of the present invention accomplishes all of the foregoing objectives and provides users with a wearer death notification bracelet that notifies at least a designated contact quickly in the event that the wearer's pulse stops for timely discovery of the deceased wearer. The device offers peace of mind to the family members and friends of people living alone, by promptly notifying them when the person living alone passes away. The device functions as a standard bracelet or wristband when it detects a pulse. The device is non-intrusive, easy to use, and can be worn comfortably and discreetly, without drawing attention or requiring the wearer to manage any complex technology. The device can be worn at all times, including during activities like bathing, where accidents are common, especially for the elderly. The device provides simple functionality of detecting a pulse and sending an alert when no pulse is detected.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a wearer death notification wearable device. The wearable device comprises a band forming a closed loop with an opening for receiving a body part such as the wrist of the wearer. The band has an inner surface and an outer surface, the inner surface being in contact with the skin of the wearer when the band is worn by the wearer. A pulse detecting sensor is embedded within the inner surface of the band and is configured to detect the pulse of the wearer. An e-sim is embedded within the band and is preconfigured with contact details of one or more third-party contacts for sending notifications upon the detection of an absence of a pulse in the user, wherein the absence of the pulse is detected continuously for a threshold time.

In another aspect, a death notification wearable device, operatively configured to notify a third-party upon the detection of the absence of a pulse in a wearer of the device is disclosed. The device includes a pulse detecting sensor integrated within the band for continuous monitoring of the wearer's pulse, wherein the sensor is a light-based sensor or an electrocardiogram sensor. An e-sim is embedded within the band, wherein the e-sim is preconfigured with contact details of at least one third-party contact for automatic notification delivery when the pulse is not detected of the wearer. A memory component is embedded within the device for storing a preconfigured time duration, wherein the sensor is configured to detect the absence of pulse for the preconfigured time duration before initiating the automatic notification to the preconfigured one or more third-party contacts.

In yet another aspect, the notification comprises pre-set and customizable content and may include a timestamp indicating when the last pulse was detected by the bracelet.

In another aspect, the wearable device functions as a conventional band when the pulse of the wearer is detected and notification is not sent.

In yet another embodiment, the wearable device includes a logo for customization and branding purposes.

In another embodiment, the sensor is a Photoplethysmogram (PPG) sensor and senses the rate of blood flow in the wearer to detect the pulse of the wearer.

In a further embodiment of the present invention, a method for sending notifications to contacts when the pulse of a wearer is not detected by a wearable device is described. The method includes providing a wearable band, the band includes a pulse detection sensor, and a means for sending notifications. The method further includes monitoring the pulse of the wearer either continuously or at regular intervals, initiating a countdown upon failure to detect a pulse, wherein the countdown progresses towards a preconfigured threshold time, and sending a notification to one or more pre-configured contacts, wherein the contacts are set during the device setup when the absence of pulse exceeds the preconfigured threshold time.

In some embodiments, the notifications sent to the third parties when the bracelet is not worn by the wearer are different from the notifications sent to the third parties when the bracelet is worn by the wearer and pulse is not detected.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the death notification wearable device of the present invention in accordance with the disclosed structure;

FIG. 2 illustrates a flow chart depicting a process of preconfiguring third-party contact details in the human pulse absence notification device of the present invention in accordance with the disclosed structure;

FIG. 3 illustrates a flow chart depicting a process of sending notification to a third-party by the death notification wearable device in accordance with one embodiment of the present invention; and

FIG. 4 illustrates a smartphone application connected to the death notification bracelet device for allowing the bracelet device to send a notification to third-party contacts in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that can be worn as a bracelet by a wearer to notify a third-party in the event of the wearer's death. Additionally, there is a long-felt need in the art for a non-intrusive wearable device that is simple to use and detects the pulse of the wearer. Moreover, there is a long-felt need in the art for a wearable device that continuously detects the pulse of a wearer and sends an alert to one or more third parties when no pulse is detected. Further, there is a long-felt need in the art for a bracelet device that offers peace of mind to family members and friends by promptly notifying them when a person wearing the bracelet passes away. Furthermore, there is a long-felt need in the art for an innovative wearable device that prevents people from being left alone for days, weeks, or even months after passing away. Finally, there is a long-felt need in the art for a bracelet device that can be worn at all times, and when detects an absence of pulse, sends a notification to one or more contacts about the passing away of the wearer.

The present invention, in one exemplary embodiment, is a death notification wearable device adapted to send notifications to one or more third parties when an absence of pulse of a wearer of the device is detected. The device includes a pulse detecting sensor for continuous or frequent monitoring of the wearer's pulse, wherein the sensor is a light-based sensor. An e-sim is preconfigured with contact details of at least one third-party contact for automatic notification delivery when the pulse is not detected of the wearer. A memory component is embedded within the device for storing a preconfigured time duration, wherein the sensor is configured to detect the absence of pulse for the preconfigured time duration before initiating the automatic notification to the preconfigured one or more third-party contacts.

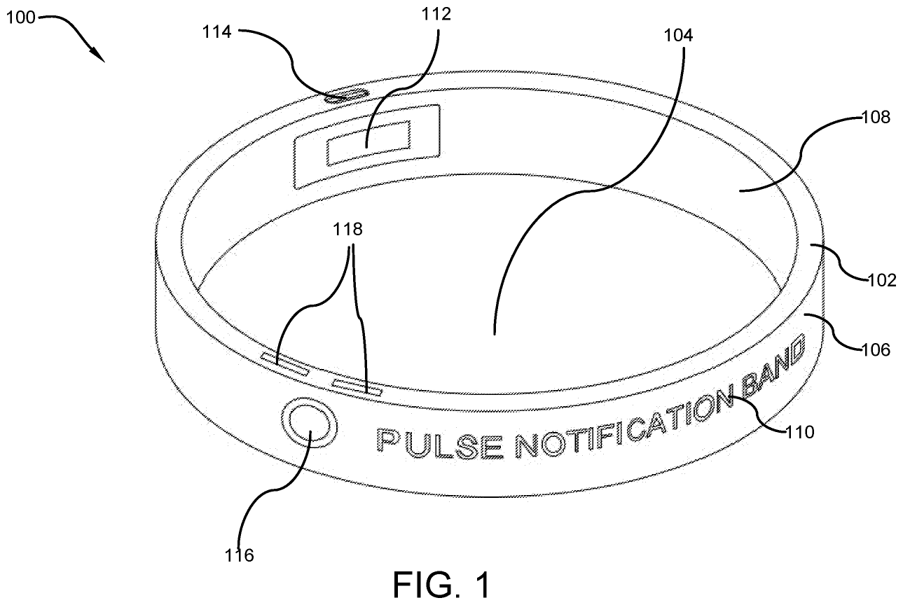

Referring initially to the drawings, FIG. 1 illustrates a perspective view of the death notification wearable device of the present invention in accordance with the disclosed structure. The death notification wearable device 100 of the present invention is designed as a wristband or bracelet for detecting the pulse of the wearer of the band device 100 and notifying at least one contact/person about the passing away of the wearer when the pulse of the wearer is not detected for a certain time period. More specifically, the death notification wearable device 100 includes a band 102 which preferably has a single-piece structure to form a closed loop defining an opening 104 for a wrist and contoured to be worn around the wrist of the wearer. The band 102 can be elastic and stretched when being worn by the wearer.

The band 102 has an outer surface 106 and an inner surface 108. The inner surface 108 touches the skin of the wearer when the death notification wearable device 100 is worn around the wrist. A logo 110 is engraved, etched, or printed on the outer surface 106 for customization or marketing purposes. The wearable device 100 includes a pulse detecting sensor 112 such as a Photoplethysmogram (PPG) sensor embedded in the inner surface 108 of the band 102 for detecting the pulse of the wearer of the band device 100. The sensor 112, for example, the PPG sensor, senses the rate of blood flow in the wearer to detect the pulse of the wearer. The sensor 112 can be a light-based sensor or can also be any other type of sensor such as an electrocardiogram sensor for detecting the pulse.

In the preferred embodiment, an e-sim 114 is embedded in the band 102 for allowing the band device 100 to send a notification to one or more third-party contacts/people as described later in the disclosure. The e-sim 114 is preconfigured with contact details of one or two contacts to which the notification is sent out when the sensor 112 does not detect the pulse of the wearer. The notification can be either audible or textual and is automatically sent.

An embedded power source 116 is included in the band 102 for providing power to the sensor 112. The power source 116 can be solar-powered and can also be replaceable. In some embodiments, the sensor 112 can be passive and may not require any external power supply. A memory 118 is embedded in the band device 100 for storing a preconfigured time for which the sensor 112 detects the absence of pulse before sending the notification to third-party contacts/people.

The band 102 can be made from one or more silicone, thermoplastic elastomer, plastic, nylon, and other similar waterproof, flexible, durable, and skin-friendly materials. Further, the human pulse detection and notification device 100 can be made in different colors, sizes, and designs to meet the requirements of different users. The death notification bracelet 100 can be worn at all times by individuals and ensures people who live alone can be identified quickly when they pass away. The e-sim size can be 2.5 mm by 2.3 mm, and the thickness can be 0.2 mm.

In some embodiments, the notifications sent to the third parties when the bracelet is not worn by the wearer can be different from the notifications sent to the third parties when the bracelet is worn by the wearer and pulse is not detected. The different notifications are configured to reduce false positives, or false negatives, and to indicate the condition of the bracelet not being worn by the wearer. The device 100 may encompass armbands, necklaces, anklets, and more.

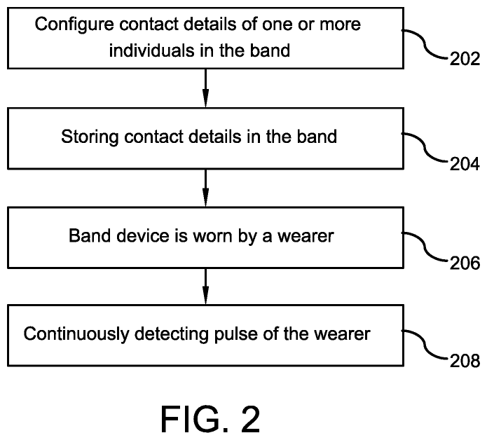

FIG. 2 illustrates a flow chart depicting a process of preconfiguring third-party contact details in the human pulse absence notification device 100 of the present invention in accordance with the disclosed structure. The death notification bracelet 100 notifies one or more designated individuals in the event of death of the wearer by monitoring the pulses of the wearer. Initially, contact details of one or more designated third-party individuals are configured in the e-sim 114 or alternatively in the memory 118 of the device 100 (Step 202). Then, the configured details are stored in the human pulse absence notification device 100 by adding the e-sim 114 and the memory 118 (Step 204). The contact details are added during purchase of the device 100 and in some embodiments can be updated remotely by the device manufacturer or using a mobile application.

Thereafter, the death notification band device 100 is worn by a wearer around the wrist (Step 206). The device 100 can be worn on any wrist and then, the sensor 112 continuously detects the pulse of the wearer (Step 208). In some embodiments, the device 100 periodically checks for a pulse of the wearer and the frequency can be adjustable or set by the manufacturer for power conservation.

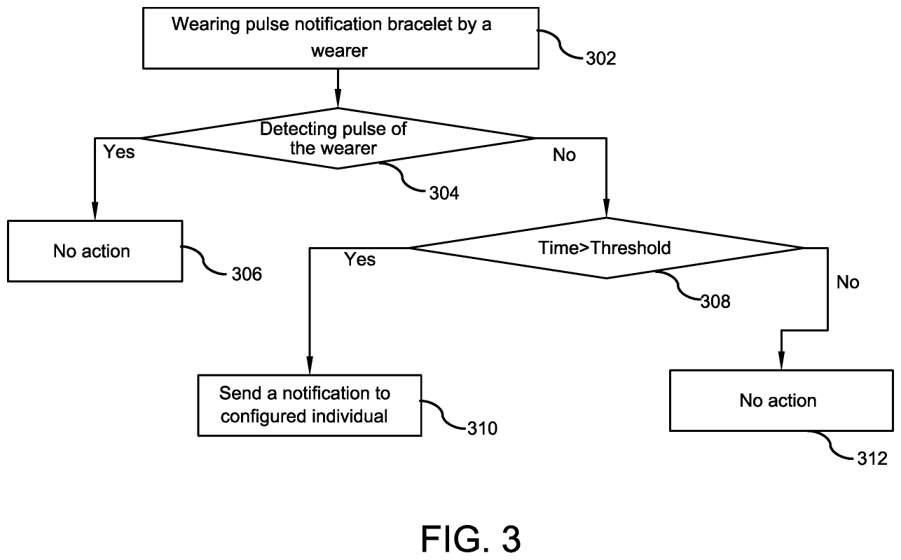

FIG. 3 illustrates a flow chart depicting a process of sending notification to a third-party by the death notification wearable device in accordance with one embodiment of the present invention. Initially, the death notification bracelet 100 is worn by a wearer and the bracelet calibrates to the normal pulse rate and other physiological parameters of the wearer (Step 302). Then, the bracelet 100 detects pulse and operates continuously or at regular intervals to monitor the wearer's pulse (Step 304). When the bracelet 100 detects the pulse, no action is performed by the bracelet, and functions as a conventional bracelet (Step 306).

When the bracelet 100 does not detect a pulse, then the bracelet starts a countdown and counts to a preconfigured threshold time (Step 308). The threshold time is set to avoid false alarms and provide correct notification. When the absence of a pulse exceeds the threshold time, the bracelet sends a notification to a pre-configured contact such as the individual during the bracelet setup process (Step 310). The notification is not sent when the time of the absence of pulse is less than the threshold time (Step 312). The notification format and content are pre-set and customizable during the setup of the bracelet and may include a timestamp when the last pulse was detected by the bracelet.

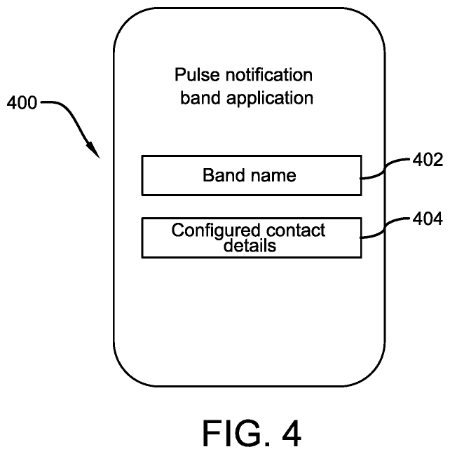

FIG. 4 illustrates a smartphone application connected to the death notification bracelet device for allowing the bracelet device to send a notification to third-party contacts in accordance with one embodiment of the present invention. In one exemplary embodiment, the bracelet device 100 does not include an e-sim but rather uses the cellular functionality of a paired smartphone application 400 for sending notifications to the third parties. As illustrated, the application 400 includes a band name 402 which is a unique identifier of the bracelet 100 with which the application 400 is paired. The application 400 pairs with only a single bracelet device 100.

The application 400 also displays the configured contact details 404 of the bracelet device 100. The application 400 allows the contact details 404 to be updated in case where the old contact details become inactive.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “death notification bracelet”, “human pulse absence notification device”, “death notification band device”, “death notification wearable device”, and “bracelet” are interchangeable and refer to the wearer pulse absence notification wearable device 100 of the present invention.

Notwithstanding the foregoing, the wearer pulse absence notification wearable device 100 of the present invention can be of any suitable size and configuration as is known in the art without affecting the overall concept of the invention, provided that it accomplishes the above-stated objectives. One of ordinary skill in the art will appreciate that the wearer pulse absence notification wearable device 100 as shown in the FIGS. is for illustrative purposes only, and that many other sizes and shapes of the wearer pulse absence notification wearable device 100 are well within the scope of the present disclosure. Although the dimensions of the wearer pulse absence notification wearable device 100 are important design parameters for user convenience, the wearer pulse absence notification wearable device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A death notification wearable device comprising:
a wristband having an outer surface and an inner surface;
a pulse detecting sensor for detecting a pulse of the wearer of said wristband;
an e-sim embedded in said wristband;
at least a first contact detail;
a power source; and
a memory;
wherein said pulse detecting sensor actuates said e-sim when a pulse is not detected for a preconfigured period of time;
wherein upon actuation said e-sim having said at least a first contact detail for automatically sending a notification to at least a first contact;
wherein said memory storing said preconfigured period of time; and
further wherein said at least a first contact is a third-party contact.

2. The death notification wearable device of claim 1 further comprising at least a second contact detail, wherein upon actuation said e-sim having said at least a first contact detail and said at least a second contact detail for automatically sending a notification to said at least a first contact and at least a second contact.

3. The death notification wearable device of claim 2, wherein said notification is selected from the group consisting of an audible notification and a textual notification.

4. The death notification wearable device of claim 3, wherein said power source is embedded in said wristband for powering said sensor.

5. The death notification wearable device of claim 4, wherein said power source is solar powered.

6. The death notification wearable device of claim 5, wherein said inner surface of said pulse detecting sensor in contact with the skin of the wearer.

7. The death notification wearable device of claim 6, wherein said wristband is elastic.

8. The death notification wearable device of claim 6, wherein said pulse detecting sensor is a Photoplethysmogram (PPG) sensor embedded in said inner surface of said wristband.

9. The death notification wearable device of claim 6, wherein said pulse detecting sensor is selected from the group consisting of a light-based sensor and an electrocardiogram sensor.

10. The death notification wearable device of claim 6, wherein said wristband having a material selected from the group consisting of a silicone, a thermoplastic elastomer, a plastic, and a nylon.

11. A death notification wearable device comprising:
a wristband having an outer surface and an inner surface;
a pulse detecting sensor for detecting a pulse of the wearer of said wristband;
an e-sim embedded in said wristband;
at least a first contact detail;
a power source; and
a memory;
wherein said pulse detecting sensor actuates said e-sim when said pulse detecting sensor stops detecting a pulse for a preconfigured period of time;
wherein upon actuation said e-sim having said at least a first contact detail for automatically sending a first notification to at least a first contact;
wherein a second notification is sent to at least a first contact when said wristband is not worn by a wearer;
wherein said first notification is different than said second notification;
wherein said memory storing said preconfigured period of time; and
further wherein said at least a first contact is a third-party contact.

12. The death notification wearable device of claim 11 further comprising at least a second contact detail, wherein upon actuation said e-sim having said at least a first contact detail and said at least a second contact detail for automatically sending at least one of said first notification and said second notification to said at least a first contact and at least a second contact.

13. The death notification wearable device of claim 12, wherein said inner surface of said pulse detecting sensor in contact with the skin of the wearer.

14. The death notification wearable device of claim 13, wherein said wristband is elastic.

15. The death notification wearable device of claim 14, wherein said pulse detecting sensor is a Photoplethysmogram (PPG) sensor embedded in said inner surface of said wristband.

16. The death notification wearable device of claim 14, wherein said pulse detecting sensor is selected from the group consisting of a light-based sensor and an electrocardiogram sensor.

17. A method of notifying a third-party of a death scenario, the method comprising the steps of:
providing a wearable device having a wristband including an outer surface and an inner surface, wherein said wristband having a pulse detecting sensor on said inner surface for detecting a pulse of the wearer of said wristband;
embedding an e-sim in said wristband, wherein said e-sim having a power source and a memory;
inputting at least a first contact detail into said e-sim for at least a first contact;
storing a preconfigured period of time in said memory;
actuating said e-sim when said pulse detecting sensor stops detecting a pulse for said preconfigured period of time;
automatically sending a first notification to said at least a first contact upon actuation of said e-sim; and
sending a second notification to said at least a first contact when said wristband is not worn by a wearer, wherein said first notification is different than said second notification.

18. The method of notifying a third-party of a death scenario of claim 17 further comprising a step of inputting at least a second contact detail into said e-sim for at least a second contact, wherein upon actuation said e-sim having said at least a first contact detail and said at least a second contact detail for automatically sending at least one of said first notification and said second notification to said at least a first contact and said at least a second contact.

19. The method of notifying a third-party of a death scenario of claim 18, wherein said inner surface of said pulse detecting sensor in contact with the skin of the wearer.

20. The method of notifying a third-party of a death scenario of claim 18, wherein said pulse detecting sensor is a Photoplethysmogram (PPG) sensor embedded in said inner surface of said wristband.
